(19) United States
(12) Patent Application Publication
(10) Pub. No.: US 2026/0347939 A1
(43) Pub. Date: Nov. 5, 2026

(54) METHODS FOR DIAGNOSING, TREATING AND PREVENTING MITRAL VALVE MYXOMATOUS DISEASE

(71) Applicant: UNIVERSITA' DEGLI STUDI DI MILANO, Milano (IT)

(72) Inventors: Cristina LECCHI, Milano (IT); Paola Giuseppina BRAMBILLA, Milano (IT); Michele POLLI, Milano (IT); Mara BAGARDI, Milano (IT); Sara GHILARDI, Milano (IT)

(21) Appl. No.: 19/468,273

(22) Filed: Feb. 3, 2026

(30) Foreign Application Priority Data
Feb. 3, 2025 (IT) 102025000001866

(51) Int. Cl. C12Q 1/6883; C12N 15/113

(52) U.S. Cl. CPC C12Q 1/6883; C12N 15/113; C12N 2310/141; C12Q 2600/178

(57) ABSTRACT

Provided are products of manufacture, biochips, arrays and miRNA panels that can be used as a prognostic marker and related methods and kits for diagnosing the risk of suffering from, the presence or absence and degree of development or severity of myxomatous mitral valve disease in dogs, by evaluating the expression levels of specific miRNAs. Provided methods for treating or ameliorating myxomatous mitral valve disease in dogs using products of manufacture, biochips, arrays and miRNA panels.

### RELATED APPLICATIONS

This U.S. utility patent application claims the benefit of priority under 35 U.S.C. § 119 (a) to Italian Patent application no. 102025000001866, filed Feb. 3, 2025. The aforementioned application is expressly incorporated herein by reference in its entirety and for all purposes.

### REFERENCE TO ELECTRONIC SEQUENCE LISTING

The application contains a Sequence Listing which has been submitted electronically in XML format and is hereby incorporated by reference in its entirety. Said .XML copy, created on Feb. 3, 2026, is named “7041-164185.xml” and is 15,245 bytes in size. The sequence listing contained in this XML file is part of the specification and is hereby incorporated by reference herein in its entirety.

### TECHNICAL FIELD

The present invention generally relates to the field of veterinary medicine and diagnostic biotechnology. In alternative embodiments, provided are products of manufacture, biochips, arrays and microRNA (miRNA) panels, which can be used as a prognostic marker, and methods and kits for diagnosing the risk of suffering from, the presence or absence of, and the degree of development or severity of myxomatous mitral valve disease (MMVD) in dogs, by assessing the expression levels of the specific miRNAs in the products of manufacture, biochips, arrays and miRNA panels as provided herein. Provided methods for treating or ameliorating myxomatous mitral valve disease in dogs using products of manufacture, biochips, arrays and miRNA panels.

### BACKGROUND

ACVIM (American College of Veterinary Internal Medicine) has proposed a classification of dogs suffering from myxomatous mitral valve disease (or myxomatous mitral valve degeneration MMVD or chronic valve degeneration) based on clinical parameters and diagnostic findings (Keene B W et al. 2019)

The dogs affected by MMVD are divided into 5 classes.

Class A identifies dogs at high risk of developing heart disease but who currently do not have an identifiable structural disorder of the heart (e.g., any Cavalier King Charles Spaniel or other predisposed breed without a heart murmur).

Class B identifies dogs with structural heart disease but who have never developed clinical signs caused by heart failure.

Class B1 describes asymptomatic dogs that exhibit echocardiographic signs of valvular degeneration in the absence of radiographic and echocardiographic signs of left atrioventricular remodeling or that echocardiographically exhibit only left atrial or ventricular remodeling.

Class B2 describes asymptomatic dogs exhibiting echocardiographic signs of valvular degeneration associated with radiographic and echocardiographic signs of left atrioventricular remodeling.

Class C indicates dogs with current or past clinical signs of heart failure caused by MMVD.

Class D refers to dogs with end-stage MMVD, where clinical signs of cardiac failure are refractory to standard treatment (defined later in this consent statement).

The aforementioned classes can also be found as called ACVIM A B B1 B2 C and D where the acronym is the ACVIM (American College of Veterinary Internal Medicine) that classified them, which is therefore analogous to the name MMVD A B B1 B2 C and D.

The microRNAs are non-coding RNA molecules involved in the regulation of gene transcription. Their presence in the blood is associated with the clinical condition of the patient, so the types and levels of circulating microRNAs vary depending on the pathophysiological status of the patient.

The identification of dogs with early asymptomatic MMVD through evaluation of miR-30b-5p that is known in tumor progression has been described (Bagardi et al., 2022).

Yang et al. (2017), using a targeted approach, described that the expression of some miRNAs carried by microvesicles, in particular cfa-miR-9, cfa-miR-181c, cfa-miR-495 and cfa-miR-599, changes with the progression of MMVD and the development of heart failure, but also changes as a function of age.

Ghilardi et al. (2022) through a statistical analysis describes a pilot study that highlights the relationship between high levels of miR-30b-5p and forms of MMVD that appear echocardiographically more stable over time in Cavalier King Charles Spaniel dogs.

Ro et al. (2021), using a targeted approach, describes that up-regulation of cfa-miR-130b is observed in dogs with stage B MMVD while cfa-miR-375 and cfa-let-7b are up-regulated in dogs with cardiac hypertrophy.

Jung and Bohan (2017) describe the characterization of circulating microRNA expression profiles by whole genome sequencing for dogs with congestive heart failure (CHF) secondary to myxomatous mitral valve degeneration (MMVD) and genetic analysis revealed distinct expression patterns of circulating microRNAs between healthy dogs and dogs with CHF, in particular, upregulation of miR-133, miR-1, miR-let-7e and miR-125 and downregulation of 4 miR-30c, miR-128, miR-142 and miR-423. Two groups of dogs referable to the classification ACVIM A (healthy and predisposed) and ACVIM C (advanced and severe form) are included in the study.

Yang et al. (2018) describes variations in miRNA expression in an in-vitro model of valvular interstitial cells (VICs) and has highlighted that VICs from diseased mitral valves show significant down-regulation of let-7c, miR-17, miR-20a and miR-30d.

Li et al 2015 describes that dogs with MMVD class B1/B2 or C/D had four up-regulated miRNAs, of which three members of the family cfa-let-7/cfa-miR-98, while seven other miRNAs were down-regulated compared to class A subjects, the expression of six of the 11 miRNAs was significantly different between dogs in phase C/D and those in phase B1/B2 and the changes in their expression were more marked with the progression of the severity of the disease.

Bagardi et al. (2022) identified only one miRNA (miR-30b-5p) as differentially expressed miRNAs in in canine MMVD, and it is not proposed as part of a biomarker panel.

Palarea-Albaladejo et al. (2024) tested several miRNAs associated with the various ACVIM stages. only one (miR-206-3p) overlaps with the present application.

Yang et al. (2018) investigated MMVD-associated miRNAs using an in vitro model based on valvular interstitial cells (VICs). This experimental approach is conceptually and technically distinct from that of the present patent application, which is based on biological fluids. MiRNAs identified in a cell model are not directly transferable or usable as in vivo biomarkers for patient screening, clinical clustering, or follow-up.

Yang et al. (2017) analyzed miRNAs carried by exosomes circulating in the blood of dogs with MMVD. Among the differentially expressed miRNAs, only one (miR-138) is in common with those of the present application. Furthermore, the authors do not classify patients according to the ACVIM guidelines, but generically divide them into “asymptomatic MMVD” and “MMVD with history of CHF”, making the comparison with the clinical stratification proposed in the present application non-overlapping.

### SUMMARY

In alternative embodiments, provided are products of manufacture, biochips, arrays, or miRNA panels, comprising, or consisting of, a plurality of miRNAs, wherein the plurality of miRNAs comprise at least the following miRNAs (optionally, at least one, 2, 3, 4, 5, 6, 7, 8, 9, 10, 11, 12, 13, 14, 15 or all 16 of):

| SEQ. | | | |
|---|---|---|---|
| ID. NO. | NAME | SEQUENCE | ST 26 STANDARD |
| SEQ. ID | cfa-miR-138b- | AGCUGGUGUUGUG | AGCTGGTGTTGTGAATCAT |
| NO. 1 | 5p | AAUCAUGCCGA | GCCGA |
| SEQ. ID | cfa-miR-144-5p | GGAUAUCAUCAUA | GGATATCATCATATACTGT |
| NO. 2 | | UACUGUAAG | AAG |
| SEQ. ID | cfa-miR-150-5p | UCUCCCAACCCUUG | TCTCCCAACCCTTGTACCA |
| NO. 3 | | UACCAGUG | GTG |
| SEQ. ID | cfa-miR-182-3p | UGGUUCUAGACUU | TGGTTCTAGACTTGCCAAC |
| NO. 4 | | GCCAACUA | TA |
| SEQ. ID | cfa-miR-206-3p | UGGAAUGUAAGGA | TGGAATGTAAGGAAGTGT |
| NO. 5 | | AGUGUGUGGG | GTGG |
| SEQ. ID | cfa-miR-207-3p | GCUUCUCCGGUCU | GCTTCTCCGGTCTCTCCTC |
| NO. 6 | | CUCCUCCUUC | CTTC |
| SEQ. ID | cfa-miR-33b-5p | GUGCAUUGCUGUU | GTGCATTGCTGTTGCATTG |
| NO. 7 | | GCAUUGC | C |
| SEQ. ID | cfa-miR-346-5p | UGUCUGCCCGCAU | TGTCTGCCCGCATGCCTGC |
| NO. 8 | | GCCUGCCUCU | CTCT |
| SEQ. ID | cfa-miR-365-1- | AGGGACUUUUGGG | AGGGACTTTTGGGGGCAG |
| NO. 9 | 5p | GGCAGAU | AT |
| SEQ. ID | cfa-miR-493-5p | UGUACAUGGUAGG | TGTACATGGTAGGCTTTCA |
| NO. 10 | | CUUUCAUU | TT |
| SEQ. ID | cfa-miR-632-5p | CUGACGGGAGCAG | CTGACGGGAGCAGAGCGG |
| NO. 11 | | AGCGGCGAA | CGAA |
| SEQ. ID | cfa-miR-8794- | AAUCGGCAAGUGG | AATCGGCAAGTGGCCTGG |
| NO. 12 | 1-5p | CCUGGGGAC | GGAC |
| SEQ. ID | cfa-miR-8875- | AUUGUCUUUGGGU | ATTGTCTTTGGGTTCTTAG |
| NO. 13 | 3p | UCUUAGCCU | CCT |
| SEQ. ID | cfa-miR-92b-3p | UAUUGCACUCGUC | TATTGCACTCGTCCCGGCC |
| NO. 14 | | CCGGCCUCC | TCC |
| SEQ. ID | cfa-miR-92b-5p | AGGGACGGGACGC | AGGGACGGGACGCGGTGC |
| NO. 15 | | GGUGCAGU | AGT |
| SEQ. ID | cfa-miR-95-3p | UUCAACGGGUAUU | TTCAACGGGTATTTATTGA |
| NO. 16 | | UAUUGAGCA | GCA |

wherein the miRNA is affixed to, or covalently or non-covalently bound to, or attached to, a surface (or at least one surface) of the product of manufacture, biochip, array, or miRNA panel.

In alternative embodiments of the product of manufacture, biochip, array, or miRNA panel as provided herein:
the plurality of miRNAs consist of at least the following miRNAs (optionally, at least one, 2, 3, 4, 5, 6, 7, 8, 9, 10, 11, 12, 13, 14, 15 or all 16 of): SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO: 15 and SEQ ID NO:16;
the plurality of miRNAs consist of the following miRNAs: SEQ ID NO: 1; SEQ ID NO:2; SEQ ID NO:3; SEQ ID NO:4; SEQ ID NO:5; SEQ ID NO: 6; SEQ ID NO:7; SEQ ID NO:8; SEQ ID NO:9; SEQ ID NO:10; SEQ ID NO: 11; SEQ ID NO:12; SEQ ID NO:13; SEQ ID NO:14; SEQ ID NO:15 and SEQ ID NO:16;
the plurality of miRNAs consist of (or consist essentially of) the following miRNAs: cfa-miR-144-5p SEQ. ID NO. 2, cfa-miR-206-3p SEQ. ID NO. 5, cfa-miR-207-3p SEQ. ID NO. 6, cfa-miR-365-1-5p SEQ. ID NO. 9, cfa-miR-493-5p SEQ. ID NO. 10, cfa-miR-632-5p SEQ. ID NO. 11;
the plurality of miRNAs consist of (or consist essentially of) the following miRNAs: cfa-miR-207-3p SEQ. ID NO. 6, cfa-miR-33b-5p SEQ. ID NO. 7, and cfa-miR-632-5p SEQ. ID NO. 11;
the plurality of miRNAs consist of (or consist essentially of) the following miRNAs: cfa-miR-144-5p SEQ. ID NO. 2, cfa-miR-150-5p SEQ. ID NO. 3, cfa-miR-182-3p SEQ. ID NO. 4, cfa-miR-206-3p SEQ. ID NO. 5, cfa-miR-365-1-5p SEQ. ID NO. 9, cfa-miR-493-5p SEQ. ID NO. 10, cfa-miR-632-5p SEQ. ID NO. 11, cfa-miR-8794-1-5p SEQ. ID NO. 12, cfa-miR-8875-3p SEQ. ID NO. 13; and/or
the plurality of miRNAs consist of (or consist essentially of) the following miRNAs: cfa-miR-138b-5p SEQ. ID NO. 1, cfa-miR-150-5p SEQ. ID NO. 3, cfa-miR-346-5p SEQ. ID NO. 8, cfa-miR-8875-3p SEQ. ID NO. 13, cfa-miR-92b-3p SEQ. ID NO. 14, cfa-miR-92b-5p SEQ. ID NO. 15, cfa-miR-95-3p SEQ. ID NO. 16.

In alternative embodiment, provided are uses of at least one miRNA, or at least 2, 3, 4, 5, 6, 7, 8, 9, 10, 11, 12, 13, 14, or 15 miRNAs, or all 16 miRNAs, wherein the miRNAs are selected from: SEQ ID NO:1; SEQ ID NO: 2; SEQ ID NO:3; SEQ ID NO:4; SEQ ID NO:5; SEQ ID NO:6; SEQ ID NO: 7; SEQ ID NO:8; SEQ ID NO:9; SEQ ID NO: 10; SEQ ID NO: 11; SEQ ID NO: 12; SEQ ID NO:13; SEQ ID NO:14; SEQ ID NO:15 and SEQ ID NO:16,
wherein the miRNAs are used as a diagnostic marker, optionally the diagnostic marker is diagnostic for myxomatous mitral valve disease, optionally diagnostic for myxomatous mitral valve disease (MMVD) in an animal of the genus Canis, and optionally the Canis is a dog or a Canis familiaris, a Canis lupus, a Canis rufus, a Canis lycaon, a Canis simensis, a Canis latrans, or a mixture thereof.

In alternative embodiment of uses as provided herein:
the detection or presence of one miRNA, or at least 2, 3, 4, 5, 6, 7, 8, 9, 10, 11, 12, 13, 14, or 15 miRNAs, or all 16 miRNAs, in a sample from a subject are diagnostic for: absence of MMVD in a subject, a healthy subject, or a subject having MMVD A;
the use provides for detection or presence of at least one miRNA, or at least three miRNAs, or all miRNAs selected from the group consisting of: cfa-miR-144-5p SEQ. ID. NO. 2, cfa-miR-206-3p SEQ. ID. NO. 5, cfa-miR-207-3p SEQ. ID. NO. 6, cfa-miR-365-1-5p SEQ. ID. NO. 9, cfa-miR-493-5p SEQ. ID. NO. 10, and cfa-miR-632-5p SEQ. ID. NO. 11, in a sample from a subject are diagnostic for MMVD B1;
the detection or presence of the miRNAs selected from the group consisting of: at least one miRNA, or at least three miRNAs, or all miRNAs, from the group consisting of: cfa-miR-207-3p SEQ. ID. NO. 6, cfa-miR-33b-5p SEQ. ID. NO. 7, and cfa-miR-632-5p SEQ. ID. NO. 11, is a diagnostic marker of MMVD B1 in subjects under 3 years of age;
the detection or presence of the miRNAs selected from the group consisting of: at least one miRNA, or at least three miRNAs, or all miRNAs from the group consisting of: cfa-miR-144-5p SEQ. ID. NO. 2, cfa-miR-150-5p SEQ. ID. NO. 3, cfa-miR-182-3p SEQ. ID. NO. 4, cfa-miR-206-3p SEQ. ID. NO. 5, cfa-miR-365-1-5p SEQ. ID. NO. 9, cfa-miR-493-5p SEQ. ID. NO. 10, cfa-miR-632-5p SEQ. ID. NO. 11, cfa-miR-8794-1-5p SEQ. ID. NO. 12, cfa-miR-8875-3p SEQ. ID. NO. 13 is diagnostic marker of MMVD B2; and/or
the detection or presence of the miRNAs selected from the group consisting of: at least one miRNA, or at least three miRNAs, or all miRNAs from the group consisting of: cfa-miR-138b-5p SEQ. ID. NO. 1, cfa-miR-150-5p SEQ. ID. NO. 3, cfa-miR-346-5p SEQ. ID. NO. 8, cfa-miR-8875-3p SEQ. ID. NO. 13, cfa-miR-92b-3p SEQ. ID. NO. 14, cfa-miR-92b-5p SEQ. ID. NO. 15, cfa-miR-95-3p SEQ. ID. NO. 16, is a discriminant diagnostic marker of MMVD B1 versus MMVD B2.

In alternative embodiments, provided are methods for diagnosing the risk of suffering, the presence or absence and the degree of development or severity of mitral valve myxomatous disease in a subject in need thereof, comprising identifying and/or measuring the expression level of at least one target miRNA, or three target miRNAs, or target miRNAs are all the miRNAs selected from the group consisting of: SEQ ID NO. 1 to SEQ ID NO. 16, in an isolated sample of a biological fluid or biopsy obtained from said subject,
wherein the presence and/or the modified level of expression of said at least one miRNA relative to a control, optionally a healthy control of the same species as the subject in need thereof, indicates an increased risk to the subject of suffering, the presence or absence, the degree of development or severity of mitral valve myxomatous disease,
wherein optionally said subject is a species of the genus Canis, or a dog.

In alternative embodiments of methods as provided herein:
the biological fluid is selected from the group consisting of: blood, serum, plasma, urine, stool, saliva;
the miRNA expression level is measured by amplification of the target miRNAs;
the control is an isolated sample obtained from a healthy subject or MMVD A or MMVD B1;
the dog is of any breed or mongrel, optionally is a Cavalier King Charles Spaniel, Chihuahua, Dachshund, Shih Tzu, Border Collie, Poodle, Maltese, Yorkshire Terrier, Whippet, Miniature Schnauzer breed, or a mixture thereof;
the methods as provided herein comprise the following steps:
a) collecting an isolated sample of biological fluid or tissue from a subject at risk or with MMVD;
b) extracting the miRNAs from the sample or tissue obtained in step a);
c) amplifying the individual miRNAs extracted in step b), preferably by quantitative PCR;
d) measuring the level of expression of at least one miRNA selected from the group consisting of: from SEQ. ID. NO. 1 to SEQ. ID. NO. 16; and
e) comparing the presence and/or the modified expression level of said at least one miRNA referred to in step d) with respect to a control wherein the presence and/or modified level of expression of at least one miRNA of step d) relative to the control indicates an increased risk to the subject of suffering from, the presence or absence, degree of development or severity of myxomatous disease of the mitral valve;
the control is an isolated sample obtained from a healthy subject or MMVD A or MMVD B1;
variation in the expression level of at least one miRNA selected from the group of: SEQ. ID. NO. 1 to SEQ. ID. NO. 16, compared to the control is indicative of the presence of the disease;
the variation in the expression level of at least one miRNA, preferably all miRNAs, selected from the group consisting of: cfa-miR-144-5p SEQ. ID. NO. 2, cfa-miR-206-3p SEQ. ID. NO. 5, cfa-miR-207-3p SEQ. ID. NO. 6, cfa-miR-365-1-5p SEQ. ID. NO. 9, cfa-miR-493-5p SEQ. ID. NO. 10, cfa-miR-632-5p SEQ. ID. NO. 11, is indicative of MMVD B1;
the variation in the expression level of at least one miRNA, preferably all miRNAs, selected from the group consisting of: cfa-miR-207-3p SEQ. ID. NO. 6, cfa-miR-33b-5p SEQ. ID. NO. 7, and cfa-miR-632-5p SEQ. ID. NO. 11, is indicative of MMVD B1 in subjects under 3 years of age;
the variation in the expression level of at least one miRNA, preferably all miRNAs, selected from the group consisting of: cfa-miR-144-5p SEQ. ID. NO. 2, cfa-miR-150-5p SEQ. ID. NO. 3, cfa-miR-182-3p SEQ. ID. NO. 4, cfa-miR-206-3p SEQ. ID. NO. 5, cfa-miR-365-1-5p SEQ. ID. NO. 9, cfa-miR-493-5p SEQ. ID. NO. 10, cfa-miR-632-5p SEQ. ID. NO. 11, cfa-miR-8794-1-5p SEQ. ID. NO. 12, cfa-miR-8875-3p SEQ. ID. NO. 13 is indicative of MMVD B2;
the variation in the expression level of all miRNAs cfa-miR-138b-5p SEQ. ID. NO. 1, cfa-miR-150-5p SEQ. ID. NO. 3, cfa-miR-346-5p SEQ. ID. NO. 8, cfa-miR-8875-3p SEQ. ID. NO. 13, cfa-miR-92b-3p SEQ. ID. NO. 14, cfa-miR-92b-5p SEQ. ID. NO. 15, cfa-miR-95-3p SEQ. ID. NO. 16, allows to discriminate MMVD to B1 subjects from MMVD B2 subjects;
cfa-miR-493-5p SEQ. ID NO. 10 is overexpressed and cfa-miR-144-5p SEQ. ID NO. 2, cfa-miR-206-3p SEQ. ID NO. 5, cfa-miR-207-3p SEQ. ID NO. 6, cfa-miR-365-1-5p and cfa-miR-632-5p SEQ. ID NO. 11 are under-expressed relative to the control;
cfa-miR-207-3p SEQ. ID. NO. 6, cfa-miR-33b-5p SEQ. ID. NO. 7, cfa-miR-632-5p SEQ. ID. NO. 11 are under-expressed compared to the control;
fa-miR-182-3p SEQ. ID NO. 4, cfa-miR-493-5p SEQ. ID NO. 10, cfa-miR-8794-1-5p SEQ. ID NO. 12, cfa-miR-8875-3p SEQ. ID NO. 13 are over-expressed and cfa-miR-144-5p SEQ. ID NO. 2), cfa-miR-150-5p SEQ. ID NO. 3, cfa-miR-206-3p SEQ. ID NO. 5, cfa-miR-365-1-5p SEQ. ID NO. 9, cfa-miR-632-5p SEQ. ID NO. 11 are under-expressed relative to the control; and/or
cfa-miR-150-5p SEQ. ID NO. 3 is under expressed and cfa-miR-138b-5p SEQ. ID NO. 1, cfa-miR-346-5p SEQ. ID NO. 8, cfa-miR-8875-3p SEQ. ID No. 13, cfa-miR-92b-3p SEQ. ID NO. 14, cfa-miR-92b-5p SEQ. ID NO. 15, cfa-miR-95-3p SEQ. ID NO. 16 are overexpressed compared to MMVD B1 stage.

In alternative embodiments, provided are kits for the in vitro evaluation of the risk of suffering from, the presence or absence and the degree of development or severity of mitral valve myxomatous disease in a subject in need thereof, wherein the kit comprises a product of manufacture, a biochip, an array, or an miRNA panel as provided herein, and optionally the kit comprises swabs and reagents for extracting, amplifying and/or measuring the expression level of at least one miRNA.

In alternative embodiments, provided are methods for treating, preventing, decreasing the symptoms of, or ameliorating myxomatous mitral valve disease (MMVD) in a subject in need thereof, comprising:
(a) identifying the subject in need thereof using a method of diagnosis of MMVD as provided herein, and
(b) treating the identified subject in need thereof with a treatment for MMVD.

In alternative embodiments of methods for treating, preventing or ameliorating MMVD:
the subject in need thereof is an animal of the genus Canis, and optionally the Canis is a dog or a Canis familiaris, a Canis lupus, a Canis rufus, a Canis lycaon, a Canis simensis, a Canis latrans, or a mixture thereof;
the MMVD treatment comprises administration of a combination of drugs comprising: pimobendan (optionally VETMEDIN™), furosemide (optionally LASIX™), an ACE Inhibitor (optionally enalapril (optionally VASOTEC™) or benazepril (optionally LOTENSIN™), spironolactone (optionally ALDACTONE™);
the MMVD treatment comprises administration of high-dose diuretics and/or sildenafil; and/or
the MMVD treatment comprises a Transcatheter Edge-to-Edge Repair or an Open-Heart Mitral Valve Repair.

In alternative embodiments, provided are methods for treating, preventing, decreasing the symptoms of, or ameliorating myxomatous mitral valve disease (MMVD) in a subject in need thereof before the subject has displayed any symptoms of MMVD, comprising:
(a) identifying the subject in need thereof using a method of diagnosis of MMVD as provided herein, and
(b) treating the identified subject in need thereof with a prophylactic treatment for MMVD.

In alternative embodiments the prophylactic treatment for MMVD comprises administration to the subject at least one, or two, or three, or four, of a combination of drugs comprising: pimobendan (optionally VETMEDIN™), furosemide (optionally LASIX™), an ACE Inhibitor (optionally enalapril (optionally VASOTEC™) or benazepril (optionally LOTENSIN™), spironolactone (optionally ALDACTONE™).

The present proposes an integrated products of manufacture, biochips, arrays and miRNA panels with clear technical applications as biomarkers; allows, in an innovative and non-obvious way, the discrimination between the ACVIM B1 and B2 stadiums, an aspect never reported before.

Therefore, although the present application uses miRNAs as biomarkers capable of discriminating ACVIM A, B, C and D stages, the proposed panel does not present significant overlaps with what is reported in the state of the art, neither from the point of view of the selected miRNAs nor from that of the claimed technical function.

10% of the canine population suffers from heart disease and, of this 10%, 75% is affected by mitral valve disease. To date, the diagnosis of mitral valve disease in dogs is carried out through a clinical evaluation by a veterinarian and an echocardiogram. The gold standard for the diagnosis of mitral valve disease in dogs is the echocardiographic examination, a very widespread, well standardized and non-invasive method that allows obtaining essential information for the correct classification of the subject and that is useful for the clinician to decide the type of drug therapy to which to subject the individual animal (Thomas et al., 1993; Keene et al., 2019; Acierno et al., 2018; Rishniw, 2018). However, this examination is often proposed when clinical signs are already present at the clinical examination, such as a heart murmur identified on auscultation, allowing to highlight lesions already developed at the valve level, without providing information regarding the possible evolution of the disease over time.

Some dog breeds, such as the Cavalier King Charles Spaniel and the Chihuahua, and, in general, small-medium dogs (under 20 kg), have sometimes very rapid evolutions, with a risk of developing heart failure even at a young age, under 4 years (Borgarelli and Buchanan, 2012). Echocardiographic examination allows the diagnosis and follow-up of these patients, but since degeneration in the early stages is hardly detectable and few veterinarians are specialized in cardiology, that is, they have the skills and experience to detect the pathology in the early stages of development, the inventors of the present invention have identified a panel of circulating microRNAs specific to mitral valve pathology in dogs and this panel of biomarkers allows an early diagnosis and staging of the pathology by means of a simple blood sample to be carried out by liquid biopsy. Moreover, thanks to the fact that they are quantifiable in the blood, they can be evaluated in a minimally invasive and repeatable way over time, thus allowing the development of the disease to be followed.

The inventors of the present invention identified and selected a novel microRNA panel by a specific screening in dogs which proved to have specific distinguishing features:
Isolated blood samples are used, so the test is minimally invasive because it is a liquid biopsy, that is, quantification in biological fluids that can be repeated over time (follow up), which allows the quantitative evaluation of the evolution of the disease;
the microRNA panel varies depending on the progress of the disease, thus allowing it to be staged in real time and quantitatively;
the microRNA panel varies long before it is possible to make the diagnosis by echocardiographic examination, thus allowing an early diagnosis of the first stage (ACVIM B1), which is hardly recognizable by a veterinarian without specialization in cardiology;
the microRNA panel allows early diagnosis (ACVIM B1) even in very young dogs under 3 years of age;
the microRNAs are very stable and resistant to degradation, therefore it is not necessary to quantify them immediately after the collection of the matrix, but it is possible to comply with the timing of a diagnostic laboratory;
molecular diagnosis through the microRNA panel allows to define more specific therapies for each patient, since it guarantees an accurate classification of patients suffering from the same pathology, also paving the way in veterinary medicine for personalized medicine.

In alternative embodiments, products of manufacture, biochips, arrays and miRNA panels as provided herein are employed as a prognostic marker and in a non-invasive diagnostic method thereof.

In alternative embodiments, products of manufacture, biochips, arrays and miRNA panels as provided herein are not derived from miRNAs known in other species, for example human and mouse, which have been translated and therefore used for diagnosis in dogs, but has been identified by characterizing the profile of miRNAs circulating in the plasma of dogs enrolled in a specific study. For this reason products of manufacture, biochips, arrays and miRNA panels as provided herein are specific to the genus canis.

The fact that the products of manufacture, biochips, arrays and miRNA panels as provided herein are based have been specifically identified in dogs, and no known miRNAs have been employed in similar diseases and in different species, means that methods as provided herein are highly specific and predictive.

In alternative embodiments, products of manufacture, biochips, arrays and miRNA panels as provided herein were identified by an OMICO approach of untargeted sequencing (NGS, Next Generation Sequencing), i.e. blood was collected from 3 groups of patients (ACVIM A, B1 and B2) and all miRNAs present in the samples were identified and quantified. Through a statistical analysis, the miRNAs differentially expressed between the 3 groups were then identified, identifying those specific to each group.

Further features of the present invention will be clear from the following detailed description with reference to the provided experimental examples.

The details of one or more exemplary embodiments of the invention are set forth in the accompanying drawings and the description below. Other features, objects, and advantages of the invention will be apparent from the description and drawings, and from the claims.

All publications, patents, patent applications, American Type Culture Collection (ATCC) deposits and NCBI reference sequences cited herein are hereby expressly incorporated by reference in their entireties for all purposes.

### DETAILED DESCRIPTION

Provided are products of manufacture, biochips, arrays and miRNA panels that can be used as a prognostic marker and related methods and kits for diagnosing the risk of suffering from, the presence or absence and degree of development or severity of myxomatous mitral valve disease in dogs, by evaluating the expression levels of specific miRNAs. Provided methods for treating or ameliorating myxomatous mitral valve disease in dogs using products of manufacture, biochips, arrays and miRNA panels.

The microRNA panel identified by the inventors of the present invention was identified by an “omical” approach of untargeted sequencing by NGS, Next Generation Sequencing, parallel sequencing, i.e. blood samples were taken from 3 groups of patients (ACVIM A, B1 and B2) and all miRNAs present in the samples were identified and quantified. Through a statistical analysis, the miRNAs differentially expressed between the 3 groups were then identified, identifying those specific to each group. The miRNA panel object of the present invention was then defined using an approach and a model different from those reported in the literature that used either a targeted approach, quantifying only some miRNAs previously reported in the literature for other heart diseases or other species, but which are not specific for dog MMVD (as in Bagardi et al., 2022; Yang et al., 2017; Ghilardi et al., 2022; Ro et al., 2021; Li et al., 2015); or an untargeted approach (not targeted), but including ACVIM A vs C groups, i.e. patients with severe and advanced mitral valve degeneration, to diagnose which it is not necessary to resort to a molecular marker (as described in Jung and Bohan, 2017). Echocardiographic examination allows the diagnosis and follow-up of patients with advanced degeneration (B2, C, and D).

Thus, since first-stage degeneration (ACVIM B1) is hardly detectable and few veterinarians are specialized in cardiology, that is, they have the skills and experience to detect the pathology in the early stages of degeneration development, the microRNA panel of the present invention is particularly useful.

Although fragmentary data relating to miRNAs in in canine MMVD are present in literature, they are different, fragmentary and not overlapping with what is the subject of the present patent application, both in terms of selected miRNAs, methodological approach, and claimed technical function.

For example Li et al. (2015) identified a panel of differentially expressed miRNAs by comparing the following clinical groups: B1/B2 versus A, C/D versus A, and C/D versus B1/B2 being different from those of the present application. Moreover, Li et al. (2015) do not carry out any analysis aimed at evaluating miRNAs as diagnostic, prognostic or clinical stratification biomarkers, limiting themselves to discussing a possible biological role as molecular modulators, and the study of Li et al. (2015) does not distinguish between ACVIM stages B1 and B2, considering stage B as a single group, without addressing the clinically relevant issue of discrimination between early preclinical and advanced preclinical stage.

In alternative embodiments, myxomatous mitral valve disease is synonymous with myxomatous mitral valve degeneration (MMVD) or chronic valve degeneration.

In alternative embodiments, MMVD class A or MMVD A means absence of degeneration of the mitral valve, therefore healthy subjects.

In the context of the present invention, MMVD class B or MMVD B means degeneration of the mitral valve characterized by structural heart disease but without clinical signs caused by heart failure.

In alternative embodiments and in the context of the present invention, MMVD class B1 or MMVD B1 means degeneration of the mitral valve characterized by lack of radiographic and echocardiographic evidence of left atrioventricular remodeling or with only left atrial or ventricular remodeling that can be evidenced echocardiographically.

In alternative embodiments and in the context of the present invention, MMVD class B2 or MMVD B2 means degeneration of the asymptomatic mitral valve that presents echocardiographic signs of valve degeneration associated with radiographic and echocardiographic signs of left atrioventricular remodeling.

In alternative embodiments variation in expression levels means under- or over-expression with respect to a control, and optionally the control is a healthy subject or an MMVD group A as provided herein.

In alternative embodiments provided are products of manufacture, biochips, arrays and miRNA panels comprising, or consisting of, the following miRNAs:

| SEQ. ID. NO. | NAME | SEQUENCE | ST 26 STANDARD |
|---|---|---|---|
| SEQ. ID NO. 1 | cfa-miR-138b- | AGCUGGUGUUGUGAAUCAUGCCGA | AGCTGGTGTTGTGAATCATGCCGA |
| | 5p | | |
| SEQ. ID NO. 2 | cfa-miR-144-5p | GGAUAUCAUCAUAUACUGUAAG | GGATATCATCATATACTGTAAG |
| SEQ. ID NO. 3 | cfa-miR-150-5p | UCUCCCAACCCUUGUACCAGUG | TCTCCCAACCCTTGTACCAGTG |
| SEQ. ID NO. 4 | cfa-miR-182-3p | UGGUUCUAGACUUGCCAACUA | TGGTTCTAGACTTGCCAACTA |
| SEQ. ID NO. 5 | cfa-miR-206-3p | UGGAAUGUAAGGAAGUGUGUGGG | TGGAATGTAAGGAAGTGTGTGG |
| SEQ. ID NO. 6 | cfa-miR-207-3p | GCUUCUCCGGUCUCUCCUCCUUC | GCTTCTCCGGTCTCTCCTCCTTC |
| SEQ. ID NO. 7 | cfa-miR-33b-5p | GUGCAUUGCUGUUGCAUUGC | GTGCATTGCTGTTGCATTGC |
| SEQ. ID NO. 8 | cfa-miR-346-5p | UGUCUGCCCGCAUGCCUGCCUCU | TGTCTGCCCGCATGCCTGCCTCT |
| SEQ. ID NO. 9 | cfa-miR-365-1- | AGGGACUUUUGGGGGCAGAU | AGGGACTTTTGGGGGCAGAT |
| | 5p | | |
| SEQ. ID NO. 10 | cfa-miR-493-5p | UGUACAUGGUAGGCUUUCAUU | TGTACATGGTAGGCTTTCATT |
| SEQ. ID NO. 11 | cfa-miR-632-5p | CUGACGGGAGCAGAGOGGOGAA | CTGACGGGAGCAGAGCGGCGAA |
| SEQ. ID NO. 12 | cfa-miR-8794- | AAUCGGCAAGUGGCCUGGGGAC | AATCGGCAAGTGGCCTGGGGAC |
| | 1-5p | | |
| SEQ. ID NO. 13 | cfa-miR-8875- | AUUGUCUUUGGGUUCUUAGCCU | ATTGTCTTTGGGTTCTTAGCCT |
| | 3p | | |
| SEQ. ID NO. 14 | cfa-miR-92b-3p | UAUUGCACUCGUCCCGGCCUCC | TATTGCACTCGTCCCGGCCTCC |
| SEQ. ID NO. 15 | cfa-miR-92b-5p | AGGGACGGGACGCGGUGCAGU | AGGGACGGGACGCGGTGCAGT |
| SEQ. ID NO. 16 | cfa-miR-95-3p | UUCAACGGGUAUUUAUUGAGCA | TTCAACGGGTATTTATTGAGCA |

In alternative embodiments products of manufacture, biochips, arrays and miRNA panels as provided herein comprise, or consist, of the following miRNAs: cfa-miR-144-5p SEQ. ID NO. 2, cfa-miR-206-3p SEQ. ID NO. 5, cfa-miR-207-3p SEQ. ID NO. 6, cfa-miR-365-1-5p SEQ. ID NO. 9, cfa-miR-493-5p SEQ. ID NO. 10, cfa-miR-632-5p SEQ. ID NO. 11.

In alternative embodiments products of manufacture, biochips, arrays and miRNA panels as provided herein comprise, or consist, of the following miRNAs: cfa-miR-207-3p SEQ. ID NO. 6, cfa-miR-33b-5p SEQ. ID NO. 7, and cfa-miR-632-5p SEQ. ID NO. 11.

In alternative embodiments products of manufacture, biochips, arrays and miRNA panels as provided herein comprise, or consist, of the following miRNAs: cfa-miR-144-5p SEQ. ID NO. 2, cfa-miR-150-5p SEQ. ID NO. 3, cfa-miR-182-3p SEQ. ID NO. 4, cfa-miR-206-3p SEQ. ID NO. 5, cfa-miR-365-1-5p SEQ. ID NO. 9, cfa-miR-493-5p SEQ. ID NO. 10, cfa-miR-632-5p SEQ. ID NO. 11, cfa-miR-8794-1-5p SEQ. ID NO. 12, cfa-miR-8875-3p SEQ. ID NO. 13.

In alternative embodiments products of manufacture, biochips, arrays and miRNA panels as provided herein comprise, or consist, of the following miRNAs: cfa-miR-138b-5p SEQ. ID NO. 1, cfa-miR-150-5p SEQ. ID NO. 3, cfa-miR-346-5p SEQ. ID NO. 8, cfa-miR-8875-3p SEQ. ID NO. 13, cfa-miR-92b-3p seq. ID NO. 14, cfa-miR-92b-5p SEQ. ID NO. 15, cfa-miR-95-3p SEQ. ID NO. 16.

In alternative embodiments, provided are methods or uses using least three miRNAs belonging to the panel consisting of the following miRNAs:

| SEQ. ID. NO. | NAME | SEQUENCE (ST 26 STANDARD) |
|---|---|---|
| SEQ. ID NO. 1 | cfa-miR-138b- | AGCTGGTGTTGTGAATCATGCC |
| | 5p | GA |
| SEQ. ID NO. 2 | cfa-miR-144-5p | GGATATCATCATATACTGTAAG |
| SEQ. ID NO. 3 | cfa-miR-150-5p | TCTCCCAACCCTTGTACCAGTG |
| SEQ. ID NO. 4 | cfa-miR-182-3p | TGGTTCTAGACTTGCCAACTA |
| SEQ. ID NO. 5 | cfa-miR-206-3p | TGGAATGTAAGGAAGTGTGTGG |
| SEQ. ID NO. 6 | cfa-miR-207-3p | GCTTCTCCGGTCTCTCCTCCTTC |
| SEQ. ID NO. 7 | cfa-miR-33b-5p | GTGCATTGCTGTTGCATTGC |
| SEQ. ID NO. 8 | cfa-miR-346-5p | TGTCTGCCCGCATGCCTGCCTC |
| | | T |
| SEQ. ID NO. 9 | cfa-miR-365-1- | AGGGACTTTTGGGGGCAGAT |
| | 5p | |
| SEQ. ID NO. 10 | cfa-miR-493-5p | TGTACATGGTAGGCTTTCATT |
| SEQ. ID NO. 11 | cfa-miR-632-5p | CTGACGGGAGCAGAGCGGCGA |
| | | A |
| SEQ. ID NO. 12 | cfa-miR-8794- | AATCGGCAAGTGGCCTGGGGA |
| | 1-5p | C |
| SEQ. ID NO. 13 | cfa-miR-8875- | ATTGTCTTTGGGTTCTTAGCCT |
| | 3p | |
| SEQ. ID NO. 14 | cfa-miR-92b-3p | TATTGCACTCGTCCCGGCCTCC |
| SEQ. ID NO. 15 | cfa-miR-92b-5p | AGGGACGGGACGCGGTGCAGT |
| SEQ. ID NO. 16 | cfa-miR-95-3p | TTCAACGGGTATTTATTGAGCA |

as a diagnostic marker, optionally a marker for myxomatous mitral valve disease, optionally in any Canis animal, such as in dogs, optionally Canis familiaris.

In alternative embodiments, products of manufacture, biochips, arrays and miRNA panels as provided herein having miRNAs selected from the group consisting of: cfa-miR-144-5p SEQ. ID. NO. 2, cfa-miR-206-3p SEQ ID NO. 5, cfa-miR-207-3p SEQ ID NO. 6, cfa-miR-365-1-5p SEQ ID NO. 9, cfa-miR-493-5p SEQ ID NO. 10, cfa-miR-632-5p SEQ ID NO. 11, are used as diagnostic markers to detect the absence of MMVD (healthy subject MMVD stage A, MMVD A).

In alternative embodiments, products of manufacture, biochips, arrays and miRNA panels as provided herein having miRNAs selected from the group consisting of: cfa-miR-144-5p SEQ. ID. NO. 2, cfa-miR-206-3p SEQ. ID. NO. 5, cfa-miR-207-3p SEQ. ID. NO. 6, cfa-miR-365-1-5p SEQ. ID. NO. 9, cfa-miR-493-5p SEQ. ID. NO. 10, cfa-miR-632-5p SEQ. ID. NO. 11, are used as diagnostic markers for MMVD at stage B1 MMVD B1.

In alternative embodiments, products of manufacture, biochips, arrays and miRNA panels as provided herein having miRNAs selected from the group consisting of: cfa-miR-207-3p SEQ. ID. NO. 6, cfa-miR-33b-5p SEQ. ID. NO. 7, and cfa-miR-632-5p SEQ. ID. NO. 11, are used as diagnostic markers for stage B1 MMVD in subjects under 3 years of age.

In alternative embodiments, products of manufacture, biochips, arrays and miRNA panels as provided herein having miRNAs selected from the group consisting of: cfa-miR-144-5p SEQ. ID. NO. 2, cfa-miR-150-5p SEQ. ID. NO. 3, cfa-miR-182-3p SEQ. ID. NO. 4, cfa-miR-206-3p SEQ. ID. NO. 5, cfa-miR-365-1-5p SEQ. ID. NO. 9, cfa-miR-493-5p SEQ. ID. NO. 10, cfa-miR-632-5p SEQ. ID. NO. 11, cfa-miR-8794-1-5p SEQ. ID. NO. 12, cfa-miR-8875-3p SEQ. ID. NO. 13 are used as diagnostic markers for MMVD at stage B2.

In alternative embodiments, products of manufacture, biochips, arrays and miRNA panels as provided herein having miRNAs selected from the group consisting of: cfa-miR-138b-5p SEQ. ID. NO. 1, cfa-miR-150-5p SEQ. ID. NO. 3, cfa-miR-346-5p SEQ. ID. NO. 8, cfa-miR-8875-3p SEQ. ID. NO. 13, cfa-miR-92b-3p SEQ. ID. NO. 14, cfa-miR-92b-5p SEQ. ID. NO. 15, cfa-miR-95-3p SEQ. ID. NO. 16, are used as diagnostic markers for MMVD at stage B1 compared to MMVD at stage B2, then discriminates stage B1 from B2.

### Methods for Treating Myxomatous Mitral Valve Disease (MMVD)

In alternative embodiments, provided are methods for treating, ameliorating, lessening the symptoms of or preventing myxomatous mitral valve disease (MMVD) in a subject in need thereof comprising first identifying the subject in need thereof using products of manufacture, biochips, arrays and miRNA panels as provided herein, the methods comprising providing a sample of biological fluid obtained from said subject and using products of manufacture, biochips, arrays and miRNA panels as provided herein to test for the presence in the sample of one, any of or all of miRNAs SEQ. ID. NO. 1 to SEQ. ID. NO. 16.

In alternative embodiments, provided are methods for diagnosing the risk of suffering from, the presence or absence and the degree of development or severity of myxomatous disease of the mitral valve which provides for the identification and/or measurement of the expression level of at least one miRNA selected from the group consisting of: SEQ. ID. NO. 1 to SEQ. ID. NO. 16, in an isolated sample of biological fluid obtained from said subject,
wherein the presence and/or modified level of expression of said at least one miRNA relative to a control, indicates an increased risk to the subject of suffering, the presence or absence, degree of development or severity of myxomatous mitral valve disease,
wherein said subject is an animal from the genus Canis, or a dog.

In alternative embodiments, provided are methods for the identification and/or measurement of the expression level of at least one, any or all miRNAs selected from the group consisting of or in the group consisting of: from SEQ. ID. NO. 1 to SEQ. ID. NO. 16.

In alternative embodiments, biological fluids or biopsies are isolated from subjects in need thereof to test for the presence of at least one, any or all miRNAs selected from the group consisting of or in the group consisting of: from SEQ. ID. NO. 1 to SEQ. ID. NO. 16, and a biological fluid or biopsy is selected from the group consisting of: blood, serum, plasma, urine, stool, saliva; blood, serum or plasma may be preferred fluid biological samples as they flow into the heart and are in direct contact with the organ involved in the disease to be investigated.

In alternative embodiments, the expression level of an miRNA in the sample is measured by amplifying target miRNAs in the sample.

In alternative embodiments a control is an isolated sample obtained from a healthy subject or MMVD A or MMVD B1 subject.

In alternative embodiments the expression level of a miRNA is evaluated with respect to the control, therefore variations in the expression levels of one or more miRNAs belonging to a miRNA panel is identifying the risk of suffering, the presence or absence and the degree of development or severity of myxomatous mitral valve disease.

In alternative embodiments the dog is of any breed or mongrel or mixed breed. In alternative embodiments the dog is less than 3 years of age. In alternative embodiments the dog is a small dog, or a subject weighing less than 20 kg.

In alternative embodiments the dog is a Cavalier King Charles Spaniel, Chihuahua, Dachshund, Shih Tzu, Border Collie, Poodle, Maltese, Yorkshire Terrier, Whippet, Miniature Schnauzer or mixed breeds of same.

In alternative embodiments methods as provided herein diagnose the risk of suffering, the presence or absence and the degree of development or severity of myxomatous mitral valve disease includes the following stages:
a) collection of an isolated sample of biological fluid from a subject at risk or with MMVD
b) extract the miRNAs from the sample obtained in stage a)
c) amplification of the individual miRNAs extracted in stage
b), preferably by quantitative PCR
d) measurement of the level of expression of at least one miRNA selected from the group consisting of: from SEQ. ID. NO. 1 to SEQ. ID. NO. 16
e) compare the presence and/or the modified expression level of said at least one miRNA referred to in step d) with respect to a control
wherein the presence and/or modified level of expression of at least one miRNA of step d) relative to the control indicates an increased risk to the subject of suffering from, the presence or absence, degree of development or severity of myxomatous disease of the mitral valve.

In alternative embodiments the control is an isolated sample obtained from a healthy subject or MMVD A or MMVD B1.

In alternative embodiments the variation in the expression level of at least one miRNA selected from the group of: SEQ. ID. NO. 1 to SEQ. ID. NO. 16, compared to the control is indicative of the presence of the disease.

In alternative embodiments the variation in the expression level of at least one miRNA selected from the group consisting of: cfa-miR-144-5p SEQ. ID. NO. 2, cfa-miR-206-3p SEQ. ID. NO. 5, cfa-miR-207-3p SEQ. ID. NO. 6, cfa-miR-365-1-5p SEQ. ID. NO. 9, cfa-miR-493-5p SEQ. ID. NO. 10, cfa-miR-632-5p SEQ. ID. NO. 11, is indicative of MMVD at the ACVIM B1 stage.

In alternative embodiments the variation in the expression level of all miRNAs: cfa-miR-144-5p SEQ. ID. NO. 2, cfa-miR-206-3p SEQ. ID. NO. 5, cfa-miR-207-3p SEQ. ID. NO. 6, cfa-miR-365-1-5p SEQ. ID. NO. 9, cfa-miR-493-5p SEQ. ID. NO. 10, cfa-miR-632-5p SEQ. ID. NO. 11 is indicative of MMVD at the ACVIM B1 stage.

In alternative embodiments the variation in the expression level of at least one miRNA selected from the group consisting of: cfa-miR-207-3p SEQ. ID. NO. 6, cfa-miR-33b-5p SEQ. ID. NO. 7, and cfa-miR-632-5p SEQ. ID. NO. 11, is indicative of MMVD at the ACVIM B1 stage in subjects under 3 years of age.

In alternative embodiments the variation in the expression level of all miRNAs cfa-miR-207-3p SEQ. ID. NO. 6, cfa-miR-33b-5p SEQ. ID. NO. 7, and cfa-miR-632-5p SEQ. ID. NO. 11 is indicative of MMVD at the ACVIM B1 stage in subjects under 3 years of age.

Subjects under 3 years of age with ACVIM B1 stage MMVD have asymptomatic early degeneration without echocardiographic evidence of left atrioventricular remodeling and with minimal valve alterations.

In alternative embodiments the variation in the expression level of at least one miRNA selected from the group consisting of: cfa-miR-144-5p SEQ. ID. NO. 2, cfa-miR-150-5p SEQ. ID. NO. 3, cfa-miR-182-3p SEQ. ID. NO. 4, cfa-miR-206-3p SEQ. ID. NO. 5, cfa-miR-365-1-5p SEQ. ID. NO. 9, cfa-miR-493-5p SEQ. ID. NO. 10, cfa-miR-632-5p SEQ. ID. NO. 11, cfa-miR-8794-1-5p SEQ. ID. NO. 12, cfa-miR-8875-3p SEQ. ID. NO. 13 is indicative of MMVD at the ACVIM B2 stage.

In alternative embodiments the variation in the expression level of cfa-miR-144-5p SEQ. ID. NO. 2, cfa-miR-150-5p SEQ. ID. NO. 3, cfa-miR-182-3p SEQ. ID. NO. 4, cfa-miR-206-3p SEQ. ID. NO. 5, cfa-miR-365-1-5p SEQ. ID. NO. 9, cfa-miR-493-5p SEQ. ID. NO. 10, cfa-miR-632-5p SEQ. ID. NO. 11, cfa-miR-8794-1-5p SEQ. ID. NO. 12, cfa-miR-8875-3p SEQ. ID. NO. 13 is indicative of MMVD at the ACVIM B2 stage.

In alternative embodiments the variation in the expression level of all miRNAs cfa-miR-138b-5p, cfa-miR-150-5p, cfa-miR-346-5p, cfa-miR-8875-3p, cfa-miR-92b-3p, cfa-miR-92b-5p, cfa-miR-95-3p, allows to discriminate MMVD subjects at ACVIM B1 stage from those at ACVIM B2 stage.

In alternative embodiments, within the characteristic panel of ACVIM B1 pathology (cfa-miR-144-5p SEQ. ID. NO. 2, cfa-miR-206-3p SEQ. ID. NO. 5, cfa-miR-207-3p SEQ. ID. NO. 6, cfa-miR-365-1-5p SEQ. ID. NO. 9, cfa-miR-493-5p SEQ. ID. NO. 10, cfa-miR-632-5p SEQ. ID. NO. 11) is overexpressed compared to the MMVD group at the ACVIM A stage cfa-miR-493-5p SEQ. ID NO. 10, while they are under-expressed with respect to the ACVIM A group: cfa-miR-144-5p SEQ. ID NO. 2, cfa-miR-206-3p SEQ. ID NO. 5, cfa-miR-207-3p SEQ. ID NO. 6, cfa-miR-365-1-5p and cfa-miR-632-5p SEQ. ID NO. 11.

In alternative embodiments, within the characteristic panel of MMVD at stage ACVIM B1 in subjects under 3 years of age: cfa-miR-207-3p SEQ. ID. NO. 6, cfa-miR-33b-5p SEQ. ID. NO. 7, and cfa-miR-632-5p SEQ. ID. NO. 11 all three are under-expressed compared to ACVIM A.

In alternative embodiments, within the characteristic panel of MMVD at the ACVIM B2 stage (cfa-miR-144-5p, cfa-miR-150-5p, cfa-miR-182, cfa-miR-206-3p, cfa-miR-365-1-5p, cfa-miR-493-5p, cfa-miR-632-5p, cfa-miR-8794-1-5p, cfa-miR-8875-3p) are overexpressed compared to ACVIM A: cfa-miR-182-3p SEQ. ID NO. 4, cfa-miR-493-5p SEQ. ID NO. 10, cfa-miR-8794-1-5p SEQ. ID NO. 12, cfa-miR-8875-3p SEQ. ID NO. 13,
are under-expressed with respect to ACVIM A: cfa-miR-144-5p SEQ. ID NO. 2), cfa-miR-150-5p SEQ. ID NO. 3, cfa-miR-206-3p SEQ. ID NO. 5, cfa-miR-365-1-5p SEQ. ID NO. 9, cfa-miR-632-5p SEQ. ID NO. 11.

In alternative embodiments, within the characteristic panel of MMVD at the ACVIM B2 stage (cfa-miR-144-5p, cfa-miR-150-5p, cfa-miR-182, cfa-miR-206-3p, cfa-miR-365-1-5p, cfa-miR-493-5p, cfa-miR-632-5p, cfa-miR-8794-1-5p, cfa-miR-8875-3p), cfa-miR-150-5p SEQ. ID NO. 3 is under-expressed with respect to the ACVIM B1 stage, while cfa-miR-138b-5p SEQ. ID NO. 1, cfa-miR-346-5p SEQ. ID NO. 8, cfa-miR-8875-3p SEQ. ID No. 13, cfa-miR-92b-3p SEQ. ID NO. 14, cfa-miR-92b-5p SEQ. ID NO. 15, cfa-miR-95-3p SEQ. ID NO. 16 are overexpressed with respect to the ACVIM B1 stage.

MMVD subjects at ACVIM B1 stage are asymptomatic patients with echocardiographically evidenced valve degeneration but who do not present echocardiographic and radiographic evidence of left atrioventricular remodeling or who present only atrial or left ventricular remodeling, while ACVIM B2 subjects are always asymptomatic and present with valve degeneration associated with both atrial and left ventricular remodeling.

In alternative embodiments provided are kits for in vitro evaluation of the risk of suffering, the presence or absence and the degree of development or severity of myxomatous disease of the mitral valve comprising swabs and reagents for extracting, amplifying and measuring the expression level of at least one miRNA selected from the group consisting of: SEQ. ID NO. 1 TO SEQ. ID NO. 16 and comparing it with a control which preferably represents a healthy subject.

In alternative embodiments, under-expression means expression of a value lower than a threshold value, which is the control, preferably healthy subject and/or ACVIM A subject.

In alternative embodiments, overexpression means expression of a higher value than the control, which is preferably the healthy subject and/or ACVIM A subject.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR-Basics: From Background to Bench, First Edition, Springer Verlag, Germany.

### Example 1

This example demonstrates that methods and compositions as provided herein using the exemplary

### Materials and Methods

### Sampling: Clinical Evaluation and Echocardiogram.

Cavalier King dogs owned and visited at the Cardiology Unit of the Department of Veterinary Medicine and Animal Science (DIVAS) of the University of Milan were enrolled in the study. In accordance with the statement of the ethical committee of the University of Milan, the informed consent was signed by the owners, number 2/2016, and a high standard of care was provided during each visit.

Cardiological evaluation was performed on dogs fasted for at least 12 hours during a routine veterinary visit. The clinical data of the animals included the medical history and clinical and echocardiographic examinations. The cardiovascular system was assessed through verification of the presence/absence of murmurs by two operators with different levels of competence, respectively a PHD student in cardiology in the third year and a professor with more than twenty years of practice in veterinary clinical cardiology. Results of auscultation included presence/absence, time and intensity of murmur (0=absent; 1=left or soft apical systolic I-II/VI; 2=bilateral or moderate and strong systolic III-IV/VI; 3=bilateral or palpable systolic V-VI/VI) (Rishniw, 2018). Blood pressure was measured indirectly by a Doppler method according to the ACVIM consensus statement (Acierno et al., 2018). Peripheral venous blood sampling was performed at the end of the examination. Blood was collected from the jugular or cephalic vein in two 2.5 ml EDTA tubes. A portion of the blood samples were used to perform routine blood tests to exclude any comorbidities. Echocardiographic examination was used to diagnose MMVD mitral valve disease. The standard transthoracic echocardiographic examination was performed with the MY LAB50 GOLD™ cardiovascular ultrasound scanner (Esaote, Genoa, Italy), equipped with multifrequency phased array probes (3.5-5 and 7.5-10 MHz), chosen according to the weight of the subject. The video clips were acquired and stored with the ultrasound software for offline measurements (Thomas et al., 1993). All tests were performed without pharmacological constraint. Dogs were classified according to the ACVIM classification scheme (Acierno et al., 2018).

The inclusion criteria for dogs in the clinically normal group (ACVIM A) were: no echocardiographic evidence of heart disease, no clinical sign, no abnormality in the results of the complete blood count and biochemical analysis, and no history of medical treatment in the previous 6 months. The inclusion criteria for dogs with stage B1 MMVD were: no abnormality in the complete blood count and biochemical analysis results, echocardiographic evidence of thickened or prolapsed mitral valve and mitral valve regurgitation, no evidence of left atrial dilatation, defined as a left atrium to aortic root ratio (LA/Ao)<1.6 on two-dimensional echocardiography, and/or no left ventricular dilatation, defined as left ventricular diastolic diameter normalized for body weight (LVIDdN)<1.7. Dogs that presented with left atrial or ventricular remodeling were also included because they did not match the criteria of the current guidelines for ACVIM class B2 (Keene et al., 2019). The degree of mitral regurgitation was assessed using Doppler color by calculating the maximum signal ratio of the regurgitant jet area to the left atrial area (Chetboul & Tissier, 2012). The size of the regurgitant jet was estimated as the percentage of the left atrial area (at the nearest 5%) occupied by the largest jet. It was considered trivial or trace (<10%), mild (between 10 and 30%), moderate (between 30 and 70%), or severe (>70%) (Chetboul & Tissier, 2012; Ponikowski et al., 2016). More specifically, mitral regurgitation was considered trivial when the regurgitant jet was not detectable in all systolic events, while it was considered trace when it was always visible (Ponikowski et al., 2016). In addition, ACVIM class B1 dogs have not had a medical treatment history in the previous 6 months. Three groups of client-owned dogs were included in this study: group A (12 subjects), or healthy control, group B1 (24 subjects); group B2 (12 subjects).

### Extraction and Sequencing of Small RNAs

Small RNAs were extracted using the MICRO RNA CONCENTRATOR™ kit (A&A Biotechnology, cat. no. 035-25C) following the manufacturer's instructions. RNA quality and quantity were verified according to MIQE guidelines (Bustin et al., 2009). RNA concentration was quantified by QUBIT® 2.0 FLUORIMETER™ with QUBIT® MICRORNA ASSAY KIT™ (Invitrogen, Cat. No. Q32880). Small RNA transcripts were converted to barcoded cDNA libraries. Library preparation was performed as previously reported (Pardini et al., 2018) using the NEBNEXT MULTIPLEX SMALL RNA LIBRARY PREP SET™ (Cat. No. NEB #E7560) for Illumina and run on NextSeq500 (Illumina Inc., USA). The output of the NEXTSEQ500 ILLUMINA SEQUENCER™ was demultiplexed using the BCL2FASTQ ILLUMINA™ software integrated into the DOCKER4SEQ™ package (Cordero et al., 2012; Pardini et al., 2018). Quantification of miRNA expression was performed using the previously described workflow (Pardini et al., 2018) and implementation as described (Beccuti et al., 2018). Sequences were mapped using SHRIMP (Rumble et al., 2009) to Canis familiaris precursor miRNAs available in MIRBASE 22.1™. Counts tables and CPM (counts per million reads) tables were used to perform differential expression (DE) analysis using as a threshold an adjusted P-value≤0.1 and an absolute log 2 Fold Change (log 2FC)≥1.

### Computational and Statistical Analysis

The quality control of raw readings, clipping of adapters, and mapping were performed as previously reported (Cordero et al., 2012). After the readings were mapped, a matrix of integer values was created. The value in the i-th row and j-th column of the matrix shows how many reads were unambiguously assigned to the mature miRNA i in sample j. The undesirable variation present in the data was estimated using the functions implemented in the SVA package (Leek et al., 2012). Differential expression analysis was performed using DESEQ2™, setting an adjusted p-value threshold <0.1 and −1≥|log 2FC|≥1. Statistical analysis was performed using SPSS 28™ (SPSS Inc., Chicago, IL, USA).

### Results

### Demographic Features

The mean age of the subjects enrolled in the study was as follows: ACVIM class A 3.1 years (min. 0.95-max. 11.3 years); ACVIM class B1 4.2 years (min. 1.1-max. 9.6 years); ACVIM class B2 8.2 years (min. 3.9-max. years). The mean body weight was: ACVIM class A 8.3 kg (min. 6.4-max. 12.4 kg); ACVIM class B1 9 kg (min. 5.8-max. 12.5 kg); ACVIM class B2 9.3 kg (min. 5.8-max. 12.1 kg). The whole females were 15, the sterilized females 10 14, the whole males 17 and castrated 2. The data for each patient are reported in Table 1, below:

TABLE 1
Data relating to the patients enrolled in the
study and classified according to ACVIM.

| Paziente | STADIO | ETÀ ANNI | PESO | SOFFIO | SESSO |
|---|---|---|---|---|---|
| 1 | A | 4.27 | 6.8 | 0 | FS |
| 2 | A | 2.05 | 8 | 0 | FS |
| 3 | A | 2.32 | 10 | 0 | F |
| 4 | A | 5.58 | 12.4 | 0 | MC |
| 5 | A | 1.04 | 6.9 | 0 | F |
| 6 | A | 0.95 | 7.4 | 0 | FS |
| 7 | A | 1.44 | 7.9 | 0 | M |
| 8 | A | 11.33 | 7 | 0 | FS |
| 9 | A | 1.28 | 9.8 | 0 | M |
| 10 | A | 3.68 | 6.4 | 0 | FS |
| 11 | A | 2.31 | 8.6 | 0 | FS |
| 12 | A | 1.23 | 8.6 | 0 | F |
| 13 | B1 | 1.16 | 8.6 | 0 | FS |
| 14 | B1 | 4.74 | 10.7 | 0 | M |
| 15 | B1 | 2.25 | 8.3 | 1 | F |
| 16 | B1 | 7.23 | 9.3 | 0 | F |
| 17 | B1 | 9.60 | 8.6 | 1 | F |
| 18 | B1 | 1.71 | 8.9 | 1 | M |
| 19 | B1 | 2.33 | 12.5 | 1 | M |
| 20 | B1 | 5.23 | 9 | 1 | FS |
| 21 | B1 | 4.73 | 10.6 | 1 | M |
| 22 | B1 | 6.30 | 9.8 | 1 | M |
| 23 | B1 | 5.40 | 7.7 | 0 | M |
| 24 | B1 | 1.54 | 7.4 | 0 | F |
| 25 | B1 | 2.28 | 8.3 | 0 | M |
| 26 | B1 | 1.88 | 9.9 | 1 | M |
| 27 | B1 | 1.77 | 8.2 | 0 | M |
| 28 | B1 | 2.04 | 8.3 | 1 | F |
| 29 | B1 | 4.57 | 7.1 | 0 | M |
| 30 | B1 | 8.35 | 9 | 1 | FS |
| 31 | B1 | 9.62 | 9.9 | 1 | FS |
| 32 | B1 | 1.36 | 5.8 | 1 | F |
| 33 | B1 | 2.05 | 10 | 0 | M |
| 34 | B1 | 5.04 | 10.5 | 1 | F |
| 35 | B1 | 8.02 | 9.5 | 1 | M |
| 36 | B1 | 2.84 | 9 | 1 | M |
| 37 | B2 | 9.31 | 12.1 | 2 | FS |
| 38 | B2 | 3.91 | 8.9 | 3 | M |
| 39 | B2 | 8.66 | 7.3 | 2 | FS |
| 40 | B2 | 7.99 | 10.8 | 1 | F |
| 41 | B2 | 7.23 | 8.1 | 2 | M |
| 42 | B2 | 9.84 | 8.6 | 2 | F |
| 43 | B2 | 9.97 | 7.1 | 2 | F |
| 44 | B2 | 8.42 | 10.6 | 3 | FS |
| 45 | B2 | 7.95 | 10.2 | 2 | F |
| 46 | B2 | 8.74 | 10.6 | 2 | F |
| 47 | B2 | 9.68 | 5.8 | 2 | FS |
| 48 | B2 | 6.38 | 12 | 2 | MC |

M = male; MC = castrated male; F = female; FS = sterilized female:

Characterization of the Circulating miRName and Identification of the Differentially Expressed miRNAs.

In order to characterize the profile of circulating miRNAs in the plasma of dogs from the ACVIM group A (12 samples), B1 (24 samples) and B2 (12 samples), sequencing was performed on the 48 samples. The number of reads for each sample ranged from 361 to 472 and 56 to 458 to 336. Samples 3, 24 and 26 were excluded from further analysis, as the number of reads mapped was too low, respectively 6017, 4009 and 3876. The counts of reads and mapped reads related to each sample are shown in Table 2.

TABLE 2
Number of reads and reads mapped relative to each sample.

| samples | sequenced | mapped | type |
|---|---|---|---|
| 1 | [symbol] | [symbol] | A |
| 2 | [symbol] | [symbol] | A |
| # 3 | [symbol] | [symbol] | A |
| 4 | [symbol] | [symbol] | A |
| 5 | [symbol] | [symbol] | A |
| 6 | [symbol] | [symbol] | A |
| 7 | [symbol] | [symbol] | A |
| 8 | [symbol] | [symbol] | A |
| 9 | [symbol] | [symbol] | A |
| 10 | [symbol] | [symbol] | A |
| 11 | [symbol] | [symbol] | A |
| 12 | [symbol] | [symbol] | A |
| 13 | [symbol] | [symbol] | B1 |
| 14 | [symbol] | [symbol] | B1 |
| 15 | [symbol] | [symbol] | B1 |
| 16 | [symbol] | [symbol] | B1 |
| 17 | [symbol] | [symbol] | B1 |
| 18 | [symbol] | [symbol] | B1 |
| 19 | [symbol] | [symbol] | B1 |
| 20 | [symbol] | [symbol] | B1 |
| 21 | [symbol] | [symbol] | B1 |
| 22 | [symbol] | [symbol] | B1 |
| 23 | [symbol] | [symbol] | B1 |
| # 24 | [symbol] | [symbol] | B1 |
| 25 | [symbol] | [symbol] | B1 |
| # 26 | [symbol] | [symbol] | B1 |
| 27 | [symbol] | [symbol] | B1 |
| 28 | [symbol] | [symbol] | B1 |
| 29 | [symbol] | [symbol] | B1 |
| 30 | [symbol] | [symbol] | B1 |
| 31 | [symbol] | [symbol] | B1 |
| 32 | [symbol] | [symbol] | B1 |
| 33 | [symbol] | [symbol] | B1 |
| 34 | [symbol] | [symbol] | B1 |
| 35 | [symbol] | [symbol] | B1 |
| 36 | [symbol] | [symbol] | B1 |
| 37 | [symbol] | [symbol] | B2 |
| 38 | [symbol] | [symbol] | B2 |
| 39 | [symbol] | [symbol] | B2 |
| 40 | [symbol] | [symbol] | B2 |
| 41 | [symbol] | [symbol] | B2 |
| 42 | [symbol] | [symbol] | B2 |
| 43 | [symbol] | [symbol] | B2 |
| 44 | [symbol] | [symbol] | B2 |
| 45 | [symbol] | [symbol] | B2 |
| 46 | [symbol] | [symbol] | B2 |
| 47 | [symbol] | [symbol] | B2 |
| 48 | [symbol] | [symbol] | B2 |

Samples indicated with # have been excluded from subsequent analyses due to the low number of reads mapped. [symbol]
indicates data missing or illegible when filed

An analysis was carried out to identify the differentially expressed miRNAs (DE-miRNAs) comparing the three ACVIM groups; the analysis identified: 15 DE-miRNAs between B1 and A (P value<0.05; −1|Log 2FoldChange|+1); 40 DE-miRNAs between B2 and A (P value<0.05; −1|Log 2FoldChange|+1); 46 DE-miRNAs between B2 and B1 (P value<0.05; −1|Log 2FoldChange|+1). Further statistical analysis was then carried out using anovaLike and Deseq2. The DE-miRNAs were a total of 16, as shown in Table 3 which shows the differentially expressed miRNAs and their sequences.

TABLE 3

| SEQ. ID. NO. | NAME | SEQUENCE | ST 26 STANDARD |
|---|---|---|---|
| SEQ. ID NO. 1 | cfa-miR-138b-5p | AGCUGGUGUUGUGAAUCAUGCCGA | AGCTGGTGTTGTGAATCATGCCGA |
| SEQ. ID NO. 2 | cfa-miR-144-5p | GGAUAUCAUCAUAUACUGUAAG | GGATATCATCATATACTGTAAG |
| SEQ. ID NO. 3 | cfa-miR-150-5p | UCUCCCAACCCUUGUACCAGUG | TCTCCCAACCCTTGTACCAGTG |
| SEQ. ID NO. 4 | cfa-miR-182-3p | UGGUUCUAGACUUGCCAACUA | TGGTTCTAGACTTGCCAACTA |
| SEQ. ID NO. 5 | cfa-miR-206-3p | UGGAAUGUAAGGAAGUGUGUGGG | TGGAATGTAAGGAAGTGTGTGG |
| SEQ. ID NO. 6 | cfa-miR-207-3p | GCUUCUCCGGUCUCUCCUCCUUC | GCTTCTCCGGTCTCTCCTCCTTC |
| SEQ. ID NO. 7 | cfa-miR-33b-5p | GUGCAUUGCUGUUGCAUUGC | GTGCATTGCTGTTGCATTGC |
| SEQ. ID NO. 8 | cfa-miR-346-5p | UGUCUGCCCGCAUGCCUGCCUCU | TGTCTGCCCGCATGCCTGCCTCT |
| SEQ. ID NO. 9 | cfa-miR-365-1-5p | AGGGACUUUUGGGGGCAGAU | AGGGACTTTTGGGGGCAGAT |
| SEQ. ID NO. 10 | cfa-miR-493-5p | UGUACAUGGUAGGCUUUCAUU | TGTACATGGTAGGCTTTCATT |
| SEQ. ID NO. 11 | cfa-miR-632-5p | CUGACGGGAGCAGAGCGGCGAA | CTGACGGGAGCAGAGCGGCGAA |
| SEQ. ID NO. 12 | cfa-miR-8794-1- | AAUCGGCAAGUGGCCUGGGGAC | AATCGGCAAGTGGCCTGGGGAC |
| | 5p | | |
| SEQ. ID NO. 13 | cfa-miR-8875-3p | AUUGUCUUUGGGUUCUUAGCCU | ATTGTCTTTGGGTTCTTAGCCT |
| SEQ. ID NO. 14 | cfa-miR-92b-3p | UAUUGCACUCGUCCCGGCCUCC | TATTGCACTCGTCCCGGCCTCC |
| SEQ. ID NO. 15 | cfa-miR-92b-5p | AGGGACGGGACGCGGUGCAGU | AGGGACGGGACGCGGTGCAGT |
| SEQ. ID NO. 16 | cfa-miR-95-3p | UUCAACGGGUAUUUAUUGAGCA | TTCAACGGGTATTTATTGAGCA |

Differentially expressed miRNAs in the various groups were also identified as shown in Table 4 which sets forth the list of the differentially expressed miRNAs in the various ACVIM and Log 2FoldChange (Log 2FC) groups relative to their expression.

Log 2FC represents the logarithm (base 2) of the ratio of expression levels between two conditions (in this case sample versus standard/control).

It can be calculated with the following formula: $$ \text{Log 2 Fold Change}=\log_2\left(\frac{text{Expression Level in Condition A}}{\text{Expression Level in Condition B}}\right) $$

Positive values indicate up-regulation, negative values indicate down-regulation while the zero value means that there is no difference in expression levels between the two conditions.

For example, conventionally Log 2FC=1 means doubled expression (hence over-expression), Log 2FC=−1: means halved expression (under-expression) and Log 2FC=0 means no change in expression.

The results are shown in Table 4:
in the ACVIM B1 group the expression of cfa-miR-493-5p SEQ. ID NO. 10 increases (Log₂ FC=1.77) compared to the ACVIM A group, while the expression of the cfa-miR-144-5p miRNAs SEQ. ID NO. 2 (Log 2 HR=−1.95), cfa-miR-206-3p SEQ. ID NO. 5 (Log 2 HR=−2.35), cfa-miR-207-3p SEQ. ID NO. 6 (Log 2 HR=−1.38), cfa-miR-365-1-5p SEQ. ID NO. 9 (Log 2 HR=−1.79), cfa-miR-632-5p SEQ. ID NO. 11 (Log 2 HR=−3.23) decreases;
in young ACVIM B1 subjects (age<3 years) three miRNAs, cfa-miR-207-3p SEQ. ID NO. 6 (Log 2FC=−1.22), cfa-miR-33b-5p SEQ. ID NO. 7 (Log 2FC=−1.78) and cfa-miR-632-5p SEQ. ID NO. 11 (Log 2FC=−1.68) decrease compared to ACVIM A;
in the ACVIM B2 group the expression of cfa-miR-182-3p SEQ. ID NO. 4 (Log 2FC=1.82), cfa-miR-493-5p SEQ. ID NO. 10 (Log 2FC=3.42), cfa-miR-8794-1-5p SEQ. ID NO. 12 (Log 2FC=12.33), cfa-miR-8875-3p SEQ. ID NO. 13 (Log 2FC=2.41) increases, while that of cfa-miR-144-5p SEQ. ID NO. 2 (Log 2FC=−3.2), cfa-miR-150-5p SEQ. ID NO. 3 (Log 2FC=−1.36), cfa-miR-206-3p SEQ. ID NO. 5 (Log 2FC=−2.51), cfa-miR-365-1-5p SEQ. ID NO. 9 (Log 2FC=−1.14), cfa-miR-632-5p SEQ. ID NO. 11 (Log 2FC=−4.02), decreases compared to the ACVIM A group;

in the ACVIM B2 group the expression of cfa-miR-150-5p SEQ. ID NO. 3 (Log 2FC=−1.23) dims, while that of cfa-miR-138b-5p SEQ. ID NO. 1 (Log 2FC=1.72), cfa-miR-346-5p SEQ. ID NO. 8 (Log 2FC=1.86), cfa-miR-8875-3p SEQ. ID NO. 13 (Log 2FC=1.82), cfa-miR-92b-3p SEQ. ID NO. 14 (Log 2FC=1.35), cfa-miR-92b-5p SEQ. ID NO. 15 (Log 2 HR=1.1), cfa-miR-95-3p SEQ. ID NO. 16 (Log 2 HR=1.49) increases compared to ACVIM B1 group.

TABLE 4

| | | | | B1 < 3 years | |
|---|---|---|---|---|---|
| | | B1 vs A | B2 vs A | vs A | B2 vs B1 |
| | miRNA | (Log₂FC) | (Log₂FC) | (Log₂FC) | (Log₂FC) |
| SEQ. ID | cfa-miR- | | | | 1.72 |
| NO. 1 | 138b-5p | | | | |
| SEQ. ID | cfa-miR- | −1.95 | −3.2 | | |
| NO. 2 | 144-5p | | | | |
| SEQ. ID | cfa-miR- | | −1.36 | | −1.2 |
| NO. 3 | 150-5p | | | | |
| SEQ. ID | cfa-miR- | | 1.82 | | |
| NO. 4 | 182-3p | | | | |
| SEQ. ID | cfa-miR- | −2.35 | −2.51 | | |
| NO. 5 | 206-3p | | | | |
| SEQ. ID | cfa-miR- | −1.38 | | −1.22 | |
| NO. 6 | 207-3p | | | | |
| SEQ. ID | cfa-miR- | | | −1.78 | |
| NO. 7 | 33b-5p | | | | |
| SEQ. ID | cfa-miR- | | | | 1.86 |
| NO. 8 | 346-5p | | | | |
| SEQ. ID | cfa-miR- | −1.79 | −1.14 | | |
| NO. 9 | 365-1-5p | | | | |
| SEQ. ID | cfa-miR- | 1.77 | 3.42 | | |
| NO. 10 | 493-5p | | | | |
| SEQ. ID | cfa-miR- | −3.23 | −4.02 | −1.68 | |
| NO. 11 | 632-5p | | | | |
| SEQ. ID | cfa-miR- | | 2.33 | | |
| NO. 12 | 8794-1-5p | | | | |
| SEQ. ID | cfa-miR- | | 2.41 | | 1.82 |
| NO. 13 | 8875-3p | | | | |
| SEQ. ID | cfa-miR- | | | | 1.35 |
| NO. 14 | 92b-3p | | | | |
| SEQ. ID | cfa-miR- | | | | 1.1 |
| NO. 15 | 92b-5p | | | | |
| SEQ. ID | cfa-miR- | | | | 1.4 |
| NO. 16 | 95-3p | | | | |

### BIBLIOGRAPHY

Acierno, M. J., et al. 2018, ACVIM consensus statement: Guidelines for the identification, evaluation, and management of systemic hypertension in dogs and cats. Journal of Veterinary Internal Medicine, 32 (6), 1803-1822.
Bagardi M, et al., 2002 Circulating MIR-30b-5p is upregulated in Cavalier King Charles Spaniels affected by early myxomatous mitral valve disease. PLOS One, July 11; 17 (7): e0266208. doi: 10.1371/journal.pone.0266208.
Beccuti, M., et al., 2018, SeqBox: RNAseq/ChIPseq reproducible analysis on a consumer game computer. Bioinformatics, 34 (5), 871-872.
Bustin, S. A., et al., 2009, The MIQE Guidelines: Minimum Information for Publication of Quantitative Real-Time PCR Experiments. Clinical Chemistry, 55 (4), 611-622.
Chetboul, V., and Tissier, R. 2012. Echocardiographic assessment of canine degenerative mitral valve disease. Journal of Veterinary Cardiology: The Official Journal of the European Society of Veterinary Cardiology, 14 (1), 127-148.
Cordero, F., et al. 2012, Optimizing a Massive Parallel Sequencing workflow for quantitative miRNA expression analysis. PLOS ONE, 7 (2).
Ghilardi S, et al. PLOS One. 2022, Prospective pilot study on the predictive significance of plasma miR-30b-5p through the study of echocardiographic modifications in Cavalier King Charles Spaniels affected by different stages of myxomatous mitral valve disease: The PRIME study. December 27; 17 (12): c0274724. doi: 10.1371/journal.pone.0274724. eCollection 2022. PMID: 36574372
Jung S, Bohan A, 2018, Genome-wide sequencing and quantification of circulating microRNAs for dogs with congestive heart failure secondary to myxomatous mitral valve degeneration. Am J Vet Res. February; 79 (2): 163-169. doi: 10.2460/ajvr.79.2.163. PMID: 29359980
Keene B W, et al., ACVIM consensus guidelines for the diagnosis and treatment of myxomatous mitral valve disease in dogs, 2019, J Vet Intern Med, 33:1127-1140.
Leek, J. T., et al. 2012, The sva package for removing batch effects and other unwanted variation in high-throughput experiments. Bioinformatics (Oxford, England), 28 (6), 882-883.
Li Q, et al., 2015, Expression Profiling of Circulating MicroRNAs in Canine Myxomatous Mitral Valve Disease. Int J Mol Sci. June 19; 16 (6): 14098-108. doi: 10.3390/ijms160614098. PMID: 26101868
Pardini, B., et al. 2018, microRNA profiles in urine by next-generation sequencing can stratify bladder cancer subtypes. Oncotarget, 9 (29).
Ponikowski, P., et al., ESC Scientific Document Group. 2016, 2016 ESC Guidelines for the diagnosis and treatment of acute and chronic heart failure: The Task Force for the diagnosis and treatment of acute and chronic heart failure of the European Society of Cardiology (ESC) Developed with the special contribution of the Heart Failure Association (HFA) of the ESC. European Heart Journal, 37 (27), 2129-2200.
Rishniw, M. 2018, Murmur grading in humans and animals: past and present. Journal of Veterinary Cardiology, 20 (4), 223-233.
Ro W B, et al., 2021, Identification and Characterization of Circulating MicroRNAs as Novel Biomarkers in Dogs With Heart Diseases. Front Vet Sci. October 11; 8:729929. doi: 10.3389/fvets.2021.729929. eCollection 2021. PMID: 34708100
Rumble, S. M., et al . . . 2009, SHRIMP: Accurate Mapping of Short Color-space Reads. PLOS Computational Biology, 5 (5), e1000386.
Thomas W. P., et al . . . 1993. Recommendations for standards in transthoracic two-dimensional echocardiography in the dog and cat. Echocardiography Committee of the Specialty of Cardiology, American College of Veterinary Internal Medicine. Journal of Veterinary Internal Medicine, 7 (4), 247-252.
Yang V K, et al. 2017, Circulating exosome microRNA associated with heart failure secondary to myxomatous mitral valve disease in a naturally occurring canine model. J Extracell Vesicles. PMID: 28804599
Yang V K, et al., 2018, Dysregulation of valvular interstitial cell let-7c, miR-17, miR-20a, and miR-30d in naturallyoccurring canine myxomatous mitral valve disease. PLOS One. January 9; 13 (1): e0188617. doi: 10.1371/journal.pone.0188617. eCollection 2018. PMID: 29315310.
Palarea-Albaladejo Javier et al., “Assessing the use of blood microRNA expression patterns for predictive diagnosis of myxomatous mitral valve disease in dogs”, Frontiers In Veterinary Science, vol. 11, 1 Nov. 2024 (2024 Nov. 1), page 1443847, Lausanne, DOI: 10.3389/fvets.2024.144384

What is claimed is:

1. A product of manufacture, a biochip, an array, or an miRNA panel, comprising, or consisting of, a plurality of miRNAs, wherein the plurality of miRNAs comprise at least the following miRNAs:

| SEQ. | | | |
|---|---|---|---|
| ID. NO. | NAME | SEQUENCE | ST 26 STANDARD |
| SEQ. ID | cfa-miR-138b- | AGCUGGUGUUGUG | AGCTGGTGTTGTGAATCAT |
| NO. 1 | 5p | AAUCAUGCCGA | GCCGA |
| SEQ. ID | cfa-miR-144-5p | GGAUAUCAUCAUA | GGATATCATCATATACTGT |
| NO. 2 | | UACUGUAAG | AAG |
| SEQ. ID | cfa-miR-150-5p | UCUCCCAACCCUUG | TCTCCCAACCCTTGTACCA |
| NO. 3 | | UACCAGUG | GTG |
| SEQ. ID | cfa-miR-182-3p | UGGUUCUAGACUU | TGGTTCTAGACTTGCCAAC |
| NO. 4 | | GCCAACUA | TA |
| SEQ. ID | cfa-miR-206-3p | UGGAAUGUAAGGA | TGGAATGTAAGGAAGTGT |
| NO. 5 | | AGUGUGUGGG | GTGG |
| SEQ. ID | cfa-miR-207-3p | GCUUCUCCGGUCU | GCTTCTCCGGTCTCTCCTC |
| NO. 6 | | CUCCUCCUUC | CTTC |
| SEQ. ID | cfa-miR-33b-5p | GUGCAUUGCUGUU | GTGCATTGCTGTTGCATTG |
| NO. 7 | | GCAUUGC | C |
| SEQ. ID | cfa-miR-346-5p | UGUCUGCCCGCAU | TGTCTGCCCGCATGCCTGC |
| NO. 8 | | GCCUGCCUCU | CTCT |
| SEQ. ID | cfa-miR-365-1- | AGGGACUUUUGGG | AGGGACTTTTGGGGGCAG |
| NO. 9 | 5p | GGCAGAU | AT |
| SEQ. ID | cfa-miR-493-5p | UGUACAUGGUAGG | TGTACATGGTAGGCTTTCA |
| NO. 10 | | CUUUCAUU | TT |
| SEQ. ID | cfa-miR-632-5p | CUGACGGGAGCAG | CTGACGGGAGCAGAGCGG |
| NO. 11 | | AGCGGCGAA | CGAA |
| SEQ. ID | cfa-miR-8794- | AAUCGGCAAGUGG | AATCGGCAAGTGGCCTGG |
| NO. 12 | 1-5p | CCUGGGGAC | GGAC |
| SEQ. ID | cfa-miR-8875- | AUUGUCUUUGGGU | ATTGTCTTTGGGTTCTTAG |
| NO. 13 | 3p | UCUUAGCCU | CCT |
| SEQ. ID | cfa-miR-92b-3p | UAUUGCACUCGUC | TATTGCACTCGTOCCGGCC |
| NO. 14 | | CCGGCCUCC | TCC |
| SEQ. ID | cfa-miR-92b-5p | AGGGACGGGACGC | AGGGACGGGACGCGGTGC |
| NO. 15 | | GGUGCAGU | AGT |
| SEQ. ID | cfa-miR-95-3p | UUCAACGGGUAUU | TTCAACGGGTATTTATTGA |
| NO. 16 | | UAUUGAGCA | GCA |

wherein the miRNA is affixed to, or covalently bound to, a surface of the product of manufacture, biochip, array, or miRNA panel.

2. The product of manufacture, biochip, array, or miRNA panel of claim 1, wherein the plurality of miRNAs consist of the following miRNAs: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; SEQ ID NO:11; SEQ ID NO:12; SEQ ID NO:13; SEQ ID NO:14; SEQ ID NO: 15 and SEQ ID NO:16.

3. The product of manufacture, biochip, array, or miRNA panel of claim 1, wherein the plurality of miRNAs consist of the following miRNAs: cfa-miR-144-5p SEQ. ID NO. 2, cfa-miR-206-3p SEQ. ID NO. 5, cfa-miR-207-3p SEQ. ID NO. 6, cfa-miR-365-1-5p SEQ. ID NO. 9, cfa-miR-493-5p SEQ. ID NO. 10, cfa-miR-632-5p SEQ. ID NO. 11.

4. The product of manufacture, biochip, array, or miRNA panel of claim 1, wherein the plurality of miRNAs consist of the following miRNAs: cfa-miR-207-3p SEQ. ID NO. 6, cfa-miR-33b-5p SEQ. ID NO. 7, and cfa-miR-632-5p SEQ. ID NO. 11.

5. The product of manufacture, biochip, array, or miRNA panel of claim 1, wherein the plurality of miRNAs consist of the following miRNAs: cfa-miR-144-5p SEQ. ID NO. 2, cfa-miR-150-5p SEQ. ID NO. 3, cfa-miR-182-3p SEQ. ID NO. 4, cfa-miR-206-3p SEQ. ID NO. 5, cfa-miR-365-1-5p SEQ. ID NO. 9, cfa-miR-493-5p SEQ. ID NO. 10, cfa-miR-632-5p SEQ. ID NO. 11, cfa-miR-8794-1-5p SEQ. ID NO. 12, cfa-miR-8875-3p SEQ. ID NO. 13.

6. The product of manufacture, biochip, array, or miRNA panel of claim 1, wherein the plurality of miRNAs consist of the following miRNAs: cfa-miR-138b-5p SEQ. ID NO. 1, cfa-miR-150-5p SEQ. ID NO. 3, cfa-miR-346-5p SEQ. ID NO. 8, cfa-miR-8875-3p SEQ. ID NO. 13, cfa-miR-92b-3p SEQ. ID NO. 14, cfa-miR-92b-5p SEQ. ID NO. 15, cfa-miR-95-3p SEQ. ID NO. 16.

7. A method for diagnosing the risk of suffering, the presence or absence and the degree of development or severity of mitral valve myxomatous disease in a subject in need thereof, comprising identifying and/or measuring the expression level of at least one target miRNA, or three target miRNAs, or target miRNAs are all the miRNAs selected from the group consisting of: SEQ ID NO. 1 to SEQ ID NO. 16, in an isolated sample of a biological fluid or biopsy obtained from said subject,
wherein the presence and/or the modified level of expression of said at least one miRNA relative to a control, optionally a healthy control of the same species as the subject in need thereof, indicates an increased risk to the subject of suffering, the presence or absence, the degree of development or severity of mitral valve myxomatous disease,
wherein optionally said subject is a species of the genus Canis, or a dog.

8. The method according to claim 7, wherein:
(a) the biological fluid is selected from the group consisting of: blood, serum, plasma, urine, stool, saliva;
(b) the miRNA expression level is measured by amplification of the target miRNAs;
(c) the control is an isolated sample obtained from a healthy subject or MMVD A or MMVD B1;
(d) the dog is of any breed or mongrel, optionally is a Cavalier King Charles Spaniel, Chihuahua, Dachshund, Shih Tzu, Border Collie, Poodle, Maltese, Yorkshire Terrier, Whippet, Miniature Schnauzer breed, or a mixture thereof;
(e) the method comprises the following steps: (i) collecting an isolated sample of biological fluid or tissue from a subject at risk or with MMVD; (ii) extracting the miRNAs from the sample or tissue obtained in step a); (iii) amplifying the individual miRNAs extracted in step b), preferably by quantitative PCR; (iv) measuring the level of expression of at least one miRNA selected from the group consisting of: from SEQ ID NO. 1 to SEQ ID NO:16; and (v) comparing the presence and/or the modified expression level of said at least one miRNA referred to in step (iv) with respect to a control, wherein the presence and/or modified level of expression of at least one miRNA of step (iv) relative to the control indicates an increased risk to the subject of suffering from, the presence or absence, degree of development or severity of myxomatous disease of the mitral valve;
(f) the control is an isolated sample obtained from a healthy subject or MMVD A or MMVD B1;
(g) variation in the expression level of at least one miRNA selected from the group of: SEQ. ID. NO. 1 to SEQ. ID. NO. 16, compared to the control is indicative of the presence of the disease;
(h) the variation in the expression level of at least one miRNA, optionally all miRNAs, selected from the group consisting of: cfa-miR-144-5p SEQ. ID. NO. 2, cfa-miR-206-3p SEQ. ID. NO. 5, cfa-miR-207-3p SEQ. ID. NO. 6, cfa-miR-365-1-5p SEQ. ID. NO. 9, cfa-miR-493-5p SEQ. ID. NO. 10, cfa-miR-632-5p SEQ. ID. NO. 11, is indicative of MMVD B1;
(i) the variation in the expression level of at least one miRNA, optionally all miRNAs, selected from the group consisting of: cfa-miR-207-3p SEQ. ID. NO. 6, cfa-miR-33b-5p SEQ. ID. NO. 7, and cfa-miR-632-5p SEQ. ID. NO. 11, is indicative of MMVD B1 in subjects under 3 years of age;
(j) the variation in the expression level of at least one miRNA, optionally all miRNAs, selected from the group consisting of: cfa-miR-144-5p SEQ. ID. NO. 2, cfa-miR-150-5p SEQ. ID. NO. 3, cfa-miR-182-3p SEQ. ID. NO. 4, cfa-miR-206-3p SEQ. ID. NO. 5, cfa-miR-365-1-5p SEQ. ID. NO. 9, cfa-miR-493-5p SEQ. ID. NO. 10, cfa-miR-632-5p SEQ. ID. NO. 11, cfa-miR-8794-1-5p SEQ. ID. NO. 12, cfa-miR-8875-3p SEQ. ID. NO. 13 is indicative of MMVD B2; and/or
(k) the variation in the expression level of all miRNAs cfa-miR-138b-5p SEQ. ID. NO. 1, cfa-miR-150-5p SEQ. ID. NO. 3, cfa-miR-346-5p SEQ. ID. NO. 8, cfa-miR-8875-3p SEQ. ID. NO. 13, cfa-miR-92b-3p SEQ. ID. NO. 14, cfa-miR-92b-5p SEQ. ID. NO. 15, cfa-miR-95-3p SEQ. ID. NO. 16, allows to discriminate MMVD to B1 subjects from MMVD B2 subjects.

9. The method of claim 8, wherein cfa-miR-493-5p SEQ. ID NO. 10 is overexpressed and cfa-miR-144-5p SEQ. ID NO. 2, cfa-miR-206-3p SEQ. ID NO. 5, cfa-miR-207-3p SEQ. ID NO. 6, cfa-miR-365-1-5p and cfa-miR-632-5p SEQ. ID NO. 11 are under-expressed relative to the control.

10. The method of claim 9, wherein cfa-miR-207-3p SEQ. ID. NO. 6, cfa-miR-33b-5p SEQ. ID. NO. 7, cfa-miR-632-5p SEQ. ID. NO. 11 are under-expressed compared to the control.

11. The method according to claim 8, wherein fa-mi-182-3p SEQ. ID NO. 4, cfa-miR-493-5p SEQ. ID NO. 10, cfa-miR-8794-1-5p SEQ. ID NO. 12, cfa-miR-8875-3p SEQ. ID NO. 13 are over-expressed and cfa-miR-144-5p SEQ. ID NO. 2), cfa-miR-150-5p SEQ. ID NO. 3, cfa-miR-206-3p SEQ. ID NO. 5, cfa-miR-365-1-5p SEQ. ID NO. 9, cfa-miR-632-5p SEQ. ID NO. 11 are under-expressed relative to the control.

12. The method according to claim 8, wherein cfa-miR-150-5p SEQ. ID NO. 3 is under expressed and cfa-miR-138b-5p SEQ. ID NO. 1, cfa-miR-346-5p SEQ. ID NO. 8, cfa-miR-8875-3p SEQ. ID No. 13, cfa-miR-92b-3p SEQ. ID NO. 14, cfa-miR-92b-5p SEQ. ID NO. 15, cfa-miR-95-3p SEQ. ID NO. 16 are overexpressed compared to MMVD B1 stage.

13. A kit for the in vitro evaluation of the risk of suffering from, the presence or absence and the degree of development or severity of mitral valve myxomatous disease,
wherein the kit comprises a product of manufacture, a biochip, an array, or an miRNA panel as set forth in claim 1,
wherein the kit optionally comprises swabs and reagents for extracting, amplifying and/or measuring the expression level of at least one miRNA.

14. A method for treating or ameliorating myxomatous mitral valve disease (MMVD) in a subject in need thereof, comprising:
(a) identifying the subject in need thereof using a method of diagnosis of MMVD claim 7, and
(b) treating the identified subject in need thereof with a treatment for MMVD.

15. The method of claim 14, wherein the subject in need thereof is an animal of the genus Canis, and optionally the Canis is a dog or a Canis familiaris, a Canis lupus, a Canis rufus, a Canis lycaon, a Canis simensis, a Canis latrans, or a mixture thereof.

16. The method according to claim 14, wherein the MMVD treatment comprises administration of a combination of drugs comprising: pimobendan (optionally VETMEDIN™), furosemide (optionally LASIX™), an ACE Inhibitor (optionally enalapril (optionally VASOTEC™) or benazepril (optionally LOTENSIN™), spironolactone (optionally ALDACTONE™).

17. The method according to claim 14, wherein the MMVD treatment comprises administration of high-dose diuretics and/or sildenafil.

18. The method according to claim 14, wherein the MMVD treatment comprises a Transcatheter Edge-to-Edge Repair or an Open-Heart Mitral Valve Repair.

19. A method for treating, preventing, decreasing the symptoms of, or ameliorating myxomatous mitral valve disease (MMVD) in a subject in need thereof before the subject has displayed any symptoms of MMVD, comprising:
(a) identifying the subject in need thereof using a method of diagnosis of MMVD of claim 7, and
(b) treating the identified subject in need thereof with a prophylactic treatment for MMVD.

20. The method of claim 19, wherein the prophylactic treatment for MMVD comprises administration to the subject at least one, or two, or three, or four, of a combination of drugs comprising: pimobendan (optionally VETMEDIN™), furosemide (optionally LASIX™), an ACE Inhibitor (optionally enalapril (optionally VASOTEC™) or benazepril (optionally LOTENSIN™), spironolactone (optionally ALDACTONE™).


SEQUENCE LISTING (16 sequences)

SEQ ID NO: 1 - RNA, 24 nt, Canis lupus
  source 1..24: mol_type = other RNA
agctggtgttgtgaatcatgccga

SEQ ID NO: 2 - RNA, 22 nt, Canis lupus
  source 1..22: mol_type = other RNA
ggatatcatcatatactgtaag

SEQ ID NO: 3 - RNA, 22 nt, Canis lupus
  source 1..22: mol_type = other RNA
tctcccaacccttgtaccagtg

SEQ ID NO: 4 - RNA, 21 nt, Canis lupus
  source 1..21: mol_type = other RNA
tggttctagacttgccaacta

SEQ ID NO: 5 - RNA, 22 nt, Canis lupus
  source 1..22: mol_type = other RNA
tggaatgtaaggaagtgtgtgg

SEQ ID NO: 6 - RNA, 23 nt, Canis lupus
  source 1..23: mol_type = other RNA
gcttctccggtctctcctccttc

SEQ ID NO: 7 - RNA, 20 nt, Canis lupus
  source 1..20: mol_type = other RNA
gtgcattgctgttgcattgc

SEQ ID NO: 8 - RNA, 23 nt, Canis lupus
  source 1..23: mol_type = other RNA
tgtctgcccgcatgcctgcctct

SEQ ID NO: 9 - RNA, 20 nt, Canis lupus
  source 1..20: mol_type = other RNA
agggacttttgggggcagat

SEQ ID NO: 10 - RNA, 21 nt, Canis lupus
  source 1..21: mol_type = other RNA
tgtacatggtaggctttcatt

SEQ ID NO: 11 - RNA, 22 nt, Canis lupus
  source 1..22: mol_type = other RNA
ctgacgggagcagagcggcgaa

SEQ ID NO: 12 - RNA, 22 nt, Canis lupus
  source 1..22: mol_type = other RNA
aatcggcaagtggcctggggac

SEQ ID NO: 13 - RNA, 22 nt, Canis lupus
  source 1..22: mol_type = other RNA
attgtctttgggttcttagcct

SEQ ID NO: 14 - RNA, 22 nt, Canis lupus
  source 1..22: mol_type = other RNA
tattgcactcgtcccggcctcc

SEQ ID NO: 15 - RNA, 21 nt, Canis lupus
  source 1..21: mol_type = other RNA
agggacgggacgcggtgcagt

SEQ ID NO: 16 - RNA, 22 nt, Canis lupus
  source 1..22: mol_type = other RNA
ttcaacgggtatttattgagca